(19) United States
(12) Patent Application Publication
(10) Pub. No.: US 2007/0060550 A1
(43) Pub. Date: Mar. 15, 2007

(54) Methods of determining compounds useful in the treatment of bipolar disorder and methods of treating such disorders

(76) Inventor: Anne Patricia White Mudge, London (GB)

Correspondence Address: LADAS & PARRY, 26 WEST 61ST STREET, NEW YORK, NY, 10023, US

(21) Appl. No.: 11/388,252

(22) Filed: Mar. 23, 2006

Related U.S. Application Data
(60) Provisional application No. 60/664,845, filed on Mar. 24, 2005.

(51) Int. Cl. A61K 31/66

(52) U.S. Cl. 514/102

(57) ABSTRACT

Compounds of use for treating bipolar disorder may be identified by tests based on their effect on various reactions involved in PIns signalling in neuronal cells. Tianeptine may be used for treatment of bipolar disorder.

### RELATED APPLICATION

This application claims priority from U.S. Patent Application Ser. No. 60/664,845, which is hereby incorporated by reference.

### FIELD OF INVENTION

The present invention relates to methods of determining compounds useful in the treatment of bipolar disorder and methods of treating such disorders.

### BACKGROUND OF THE INVENTION

Numerous aspects of bipolar disorders, formerly referred to as manic depression, are only slowly becoming understood.

Bipolar disorder occurs in about 1.5% of the population world-wide. The condition can involve either discrete periods of depression and mania or rapid cycling between these extremes. These characteristics have led to the use of mood stabilizing drugs for treatment of bipolar disorder. The normal prophylactic treatment has been with lithium and in more recent years with valproic acid (or a valproate salt, normally the sodium salt) and carbamazepine. All of these are effective in the control of mania and both lithium and valproic acid are effective at limiting the frequency of depressive episodes. However none of these drugs is used to treat acute depression, which tends to be treated with conventional antidepressants. Unfortunately, such antidepressants can trigger mania or increase the frequency of mood swings, which can be problematical for a patient suffering from bipolar disorder. The problem is compounded by the fact that there is often a long delay between the onset of the condition and its diagnosis.

Furthermore, the drugs currently in use to treat bipolar disorder may have unacceptable side effects, and the need to be able to identify new drugs for treatment of bipolar disorders remains. Moreover, the aforementioned drugs are all more effective in preventing episodes of mania rather than of depression, whereas the extent of the burden of depression in this disorder has been recently high-lighted (Goodwin, 2003; Post et al., 2003a). The depressive pole of this disorder is usually treated with conventional antidepressant drugs developed to treat unipolar depression (Post et al., 2003b), although the anticonvulsant drug lamotrigine has been shown recently to be effective in treating various types of bipolar depression (Calabrese et al., 2002b). None of these drugs was developed with a particular theory of the underlying biochemical basis of bipolar disorder in mind, but rather existing drugs were tested for their ability to control bipolar disorder: note that all of VPA, CBZ, and lamotrigine are also used to treat epilepsy (Calabrese et al., 2002a).

In a recent article by A. J. Harwood Molecular Psychiatry (2004) 10 117-126 (Harwood, 2005), it is noted that the three most commonly prescribed drugs used to treat this disorder—lithium, valproic acid, and carbamazepine—each have an effect on intracellular signalling pathways within cells. In his review, Harwood notes that it has been known for over twenty years that lithium inhibits the enzymes inositol polyphosphate-1-phosphatase (IPPase) and inositol monophosphatase (IMPase), which both control recycling of myo-inositol (sometimes referred to simply as inositol) following phosphatidylinositide breakdown and that lithium also reduces the cellular concentration of inositol in the brain. The review notes that a number of compounds based on an inositol backbone, including the membrane inositol phospholipids collectively known as phosphatidylinositides (PtdIns)—or as phosphoinositides (PIns)—play a key role in intracellular signalling pathways. Signalling via phosphatidyl inositol-4,5-bisphosphate (PIP₂) may be very important in signalling events at synapses in the brain (see Osborne et al., 2001). Inositol itself is both supplied in the diet and synthesized within some cells from glucose-6-phosphate by the mammalian enzyme known as myo-inositol phosphate synthase (MIP-synthase); in yeast the equivalent enzyme is known as Ino-1 (Ju et al., 2004). The Harwood article (2005) notes that valproate inhibits the enzyme MIP-synthase in brain (Shaltiel et al., 2004) and MIP-synthase has been suggested as a potential target for the development of new drugs to treat bipolar disorder (Agam et al., 2002). The Harwood article notes that all of lithium, valproic acid and carbamazepine affect the transport of extracellular myo-inositol into cells, notably astrocytes in brain and also astrocyte cell lines, and the high-affinity sodium/myo-inositol transporter (SMIT) has also been suggested as a target for the development of new drugs to treat bipolar disorder (van Calker and Belmaker, 2000). MIP-synthase expression is confined to the vasculature in brain (Wong et al., 1987); SMIT-1 is expressed predominently in the choroid plexus (Guo et al., 1997) and is up-regulated by injury, suggesting that it may be involved in regulating the osmotic effects of inositol in the brain.

The Harwood article (2005) goes on, however, to point to the possibility that the degree of myo-inositol-depletion is not a significant effect in brain—being usually of the order of a 50% reduction in brain levels. Harwood goes on to suggest that the action of lithium and valproic acid in mood stabilization may result from their interaction with other cellular targets. Having discussed these issues, Harwood concludes all of these problems have so far prevented complete testing of the inositol-depletion hypothesis as presented by Berridge, Downes and Hanley in 1989 (Berridge et al., 1989) and that it is still impossible to draw conclusions to its clinical relevance. That said, he notes that the hypothesis remains viable and new discoveries, such as inositol-depletion effects of other mood stabilizers, the role of sodium myo-inositol transporters (SMIT 1 and 2) and the “intriguing associated prolyl oligopeptidase (PO) activity” have augmented the hypothesis since its original inception. He notes that continued work at the basic, preclinical and clinical level may yet establish whether changes in inositol phosphate signaling form the basis of mood stabilizer therapy and perhaps the origins of bipolar disorders.

Shaltiel et al. in Biol. Psychiatry 2004; 56; 868-874 indicate that common features of drugs to treat bipolar disorder are 1) increased spreading of growth cones of rat dorsal root ganglia cells, 2) 4871 reduction of inositol synthesis. They report their view that VPA effects the latter by affecting MIP-synthase. I do not believe this to be correct but, as noted below, that VPA's effect on limiting the frequency of mania derives from its inhibition of phosphoinositide (PIns) synthesis, whilst its effect on limiting depression derives from its stimulation of PIns synthesis, perhaps by inhibiting PO.

A possible mechanism that links PIP₂ synthesis with transport of inositol into the cell has been described recently: The H⁺/myo-inositol transporter (HMIT) is inserted into the plasma membrane in an activity-dependent manner (Uldry et al., 2004), but no mention was made of this transporter as a possible drug target. In another article, Uldry has commented that an impaired inositol mechanism has been linked to psychiatric diseases, in particular bipolar disorder, suggesting that inhibition of HMIT could lead to beneficial effects for bipolar disorders by decreasing intracellular inositol concentrations Uldry et al. (2004) The SLC2 family of facilitated hexose and polyol transporters Eur. J. Physiol 447:480. Uldry has also described the cloning and functional characterization of this novel HMIT, noting that it has the same membrane topology as certain glucose transporters but noting a long extracellular loop containing three N-glycosylation sites and the fact that the HMIT did not share any amino acid homology with sodium-dependent transporters (SMIT) that also act to transport inositol.

In a letter to Nature (2002) (417, 292-295) 1 and a number of co-workers (Williams et al., 2002) reported that while the underlying mechanism of the action of lithium, valproic acid and carbamazepine in the treatment of bipolar disorder was unknown, we found that all three of the drugs inhibit the collapse of sensory neuron growth cones and increase growth cone area in these neurons grown in culture and that these effects were reversed by addition of exogenous inositol and certain inhibitors of the activity of the enzyme prolyl oligopeptidase (PO). I used the growth cones as a model system for assessing changes in intracellular signalling events in response to drugs: The growth cones have some properties in common with presynaptic nerve terminals in the brain. I argued that, if the signalling changes we see in the growth cones of cultured neurons also occur in mature brain synapses in mood-related circuits, then these changes in neuronal signalling in response to the drugs would very likely affect synaptic transmission and thus also affect mood.

Inhibitors of prolyl oligopeptidase have themselves been reported to increase the basal levels of the second messenger signaling molecule inositol-1,4,5-trisphosphate (InsP₃) by about 2-fold (Schulz et al., 2002; Williams et al., 1999). It is not clear, however, how a 2-fold change in basal InsP₃ would affect the ability of a neuron to signal because the change in levels of (InsP₃) in response to an extracellular signal is usually in the order of more than 10-fold and is also short-lived of the order of seconds. Moreover, an important aspect of PtdIns signaling in neurons is the ability of phosphatidylinositol4,5-bisphosphate (PIP₂) to bind proteins important for synaptic function (see review Osborne et al., 2001). Nevertheless, although such disclosures may suggest a link between prolyl oligopeptidase and InsP₃ signalling, this has not hitherto been generally accepted as proven.

Although the inositol phosphate signaling pathway in neurons has been suggested as playing a key role in mood control, no explanation has been given as to how such signaling relates to the control of both the depressive and manic aspects of bipolar disorder (Harwood, 2005). It has also been noted, however, that the other signaling pathways may also be relevant to mood control and to the therapeutic action of the mood-stabilizing drugs: for example, there is some evidence suggesting that the therapeutic target of lithium may instead be glycogen synthase kinase-3 (GSK-3) and that the therapeutic target of valproic acid may be histone deacetylase (HDAC) (see, for example the reviews by (Gould et al., 2004; Gurvich and Klein, 2002; Harwood and Agam, 2003). In all of these reviews, none of the suggested targets of the mood stabilizers provide a mechanism for the ability of lithium and valproic acid to limit mood swings to both manic and depressive episodes. A recent paper from Klein and colleagues (O'Brien et al., 2004) does link decreased levels of GSK-3 gene expression to antidepressant-like behavioural changes, however, and this GSK-3 effect may contribute to the ability of lithium to limit mood swings to depression.

The depressive pole of bipolar disorder is often treated with conventional antidepressant drugs, but these tend to destabilize the illness and there are suggestions that the antidepressant drugs may induce switching to mania or hypomania (Post et al., 2001; Post et al., 2003b). The anticonvulsant drug, lamotrigine, has been reported to be efficacious in treating bipolar depression with less tendency to induce manic symptoms, but lamotrigine has side-effects that may limit its use in bipolar disorder (Calabrese et al., 2002b; Calabrese et al., 2002c). I am not aware of reports in the literature suggesting that conventional antidepressant drugs may exert their therapeutic effects by increasing synthesis of PIP₂ or increasing the availability of myo-inositol for the synthesis or recycling of phosphoinositides such as PIP₂. Instead, conventional antidepressants such as desipramine and fluoxetine are thought to exert their effects by initially inhibiting the reuptake of monoamines into nerve terminals (Berton and Nestler, 2006).

The effects of lithium, valproic acid and carbamazepine on inositol-dependent signaling in cultured neurons that I reported with my colleagues (Williams et al., 2002) is to date the only common mechanism of action of these three drugs that could explain their therapeutic action on neuronal circuits. Our report indicated that prolyl oligopeptidase inhibitors antagonize the action of the commonly prescribed mood stabilizers, thereby teaching against their use in treatment of bipolar disorder or depression. A problem with the results we reported, however, is that they only offer an explanation for one pole of bipolar disorder, most likely the manic pole. (Carbamazepine is mainly an antimanic drug having no clear benefit in controlling the depressive phase of bipolar disorder.)

However, I now think that the action of all three mood-stabilizing drugs-lithium, valproic acid, and carbamazepine-on the morphology of sensory neuron growth cones involves inhibition of phosphoinositide (PIns) synthesis following breakdown of phosphatidylinositol-4,5-bisphosphate (PIP₂) in the membrane and that the resynthesis of PIP₂ is dependent on the local supply of inositol to the site of synthesis in the neuronal membranes rather than to significant depletion of total inositol levels within the neuron. The local supply of inositol may result from either inhibiting recycling from InsP₃ or by inhibiting transport of inositol into neurons from external sources.

I have now found that inhibitors of PO can reverse the inhibitory effects of lithium on the recycling of PIns thus providing evidence that inhibitors of PO normally have a stimulatory effect on the rate of PIns recycling: This provides the first evidence that inhibitors of PO can stimulate PIns signalling in contrast to previous results concerning effects of PO on basal levels of InsP₃ discussed above. In this context I have now surprisingly found that valproic acid (VPA) inhibits prolyl oligopeptidase activity (PO) directly at a dose that is compatible with therapeutic blood levels, which is usually of the order of 0.3-0.7 mM. This result was unexpected since our prior work led to the expectation that VPA decreases phosphoinositide synthesis and signalling, but inhibition of PO by VPA should lead to an increase in phosphoinositide synthesis and signalling. I have discussed this new finding—that VPA inhibits PO directly with a Kᵢ of ˜1 mM—in the context of the ability of VPA to limit mood swings to both mania and depression (see Cheng, Lumb, Polgar and Mudge-Molecular and Cellular Neuroscience 29, 155-161, 2005) (Cheng et al., 2005): I propose in that paper that depression may involve a less than optimal level of phosphoinositide production and signalling in neurons in mood-related circuits.

This proposal is based on my other new findings, namely that conventional antidepressants (ADs), such as desipramine and fluoxetine, can reverse the inhibitory effects of lithium, valproic acid and carbamazepine on the PIns cycle. The ADs act at micromolar concentrations, which is a level reached in the brain during treatment with these drugs. Interestingly, I unmasked this new action of the ADs by first perturbing the cycle with the mood stablilizers, which lowers PIns signalling, as described in Williams et al. 2002. My results suggest methods for screening for new drugs to control bipolar depression, namely to screen for drugs that stimulate the PIns cycle and whose action is maximal when PIns signalling is less than optimal (see diagram in FIG. 2). Conversely, when screening for new drugs to control the manic phase of the disorder, the search should be for drugs that inhibit the PIns cycle and have their maximal effect when there is increased activity in the cycle. (Note that inhibiting HMIT fits the latter). Moreover, an ideal mood stabilizer should, like VPA, be able to affect both the highs and the lows of mood states by its ability to limit both the highs and lows of PIns signalling. Alternatively, combinations of drugs that act on each pole could be given to stabilize mood from both above and below the norm. The advantage of screening for new drugs in this way would be that they are screened specifically for targeting PIns activity without having the multiple activities that other drugs have.

My proposal has been confirmed by my recent findings with lamotrigine, which as noted previously has now started to be used for treatment of bipolar disorder, and has its main effect on the depressive pole of this illness without increases the frequency of mood swings to mania (Muzina et al., 2005). The mechanism of action for lamotrigine to act as a mood stabilizer and as a antidepressant drug is not known (Ketter et al., 2003). I now find that lamotrigine has an effect on growth cones similar to the mood stabilizers lithium, valproic acid, and carbamazepine, in that it inhibits the collapse of growth cones and this effect is reversed by increasing the extracellular concentration of inositol. Moreover, I found that lamotrigine acts in similar fashion to desipramine in that it can reverse the action of either carbamazepine or valproic acid on the growth cones, presumably by stimulating the PIns cycle. The dual effects of lamotrigine on the growth cones I now report confirms my prediction that an ideal mood stabilizer would have dual effects on the PIns recycling by being able to control both the highs and lows of PIns signalling. In this way it could control bipolar depression without increasing the frequency of mania or destabilizing mood.

I obtained further support for the involvement of PIns signalling in the action of antidepressant drugs from my study of two other drugs that are used clinically to treat depression, namely olanzapine; this is an atypical antipsychotic drug that acts as an antidepressant in schizophrenia and bipolar disorder (Tohen et al., 2003).

Without wishing to be bound by any theory, I believe that control of bipolar disorder requires the maintenance of optimal levels of PIns signaling in neurons in mood-related circuits, particularly in the cerebral cortex. When such signalling exceeds the norm the person's mood tends to become manic. When PIns signalling is below the norm the person's mood is depressed. The dual ability of lamotrigine to act as both an antidepressant-like and a mood stabilizer-like (antimanic drug) compound supports my theory that screening for these dual effects in PIns signalling will lead to drugs useful for the treatment of both poles of bipolar disorder. Such drugs with effective antidepressant activity in addition to an inhibitory effect on PIns recycling should not increase the frequency of mood swings to mania as do the conventional antidepressants such as desipramine and fluoxetine.

When PIns signaling is below the norm the person's mood is depressed. As noted above, a limit on the supply of inositol used to synthesize PIP₂ would lead to reduced PIns signaling. Reduction in the supply of inositol used to synthesize PIP₂ in neurons is therefore appropriate to control elevated PIns signaling associated with mania. Such reduction in the supply of inositol could be achieved either by inhibiting the enzymes involved in recycling inositol from cytosolic inositol polyphosphates (such as seen with lithium's inhibition of IMPase and IPPase) or by limiting the transport of inositol into the neuron from the extracellular space, but perhaps not by inhibiting the transporters SMIT 1 and 2. Alternatively, limiting the supply of phosphatidic acid (PA) used to synthesize PIns by perhaps inhibiting DAG-kinase would also be appropriate to control mania as would inhibiting the kinases that convert PI to PIP and then to PIP₂. (See FIG. 1 for a diagram of the phosphoinositide cycle that also shows the enzymes known to be involved in recycling of InsP₃ to inositol and in the synthesis of PIP₂ from inositol.)

Control of the depressed mood on the other hand requires increased PIns synthesis and signaling: Inhibition or activation of enzymes that result in increased synthesis of PIns is therefore likely to elevate depressed mood. In particular one means of increasing the availability of inositol or increasing the synthesis of PIP₂ may be to inhibit the activity of phosphatidic acid phosphatase (PAPase). Another means of increasing the availability of inositol would be to decrease the generation of additional forms of inositol phosphates that normally divert recycling of InsP₃ through the known pathways. In this context, we have recently found an abundant and unusual form of inositol phosphate in cultured cortical neurons that may play a role in the supply of inositol for PIns recycling.

Another means of increasing the synthesis of PIP₂ would be to inhibit the activity of the enzyme prolyl oligopeptidase (PO): we showed in Williams et al., (2002) that certain inhibitors of prolyl oligopeptidase have an effect that is equivalent to adding exogenous inositol to the cultured neurons. U.S. Pat. No. 5,384,322 describes inter alia the prolyl oligopeptidase inhibitor (2S, 3aS, 7aS)-1 {[(RR-2 phenylcyclopropyl]carbonyl}-2-[(thiazolidine-3-yl) carbonyl]octahydro-1H-indole and suggests its use for treatment of memory impairment, and Alzheimer's disease and depression. This compound has been tested under the designation S17092-1.

Prolyl oligopeptidase is an unusual serine protease that has a beta-propeller structure, which controls access of the proteolytic substrate to the catalytic site (Fulop et al., 1998). Since the beta-propeller type of structure is shared by many proteins that act via protein/protein interactions, it is possible that PO has regulatory functions other than its proteolytic function. Inhibitors that affect either the catalytic function or putative regulatory functions controlled by protein-protein interactions and/or the beta-propeller may be useful as therapeutic agents.

Work underlying this invention has been funded by grants from the Meducal Research Council (UK) and the Stanley Medical Research Institute.

### SUMMARY OF THE INVENTION

From a first aspect the present invention produces a method for treating bipolar disorders that comprises treating a patient suffering therefore with a therapeutically effective amount of a cell permeable compound (other than valproic acid and lithium) or a combination of compounds that is or are capable of inhibiting the activity of prolyl oligopeptidase within neurons.

From a second aspect the invention provides a means for screening a compound or combination of compounds for their suitability for use in treatment of bipolar disorder by testing for their ability to 1) inhibit the activity of prolyl oligopeptidase within neurons and 2) stimulate the biochemical pathway for production or recycling of phosphatidylinositides such as PI, PIP and PIP₂ and selecting a compound or combination of compounds effective to do both. The compound should ideally be most active when activity in the PIns cycle is low.

From a third aspect the present invention produces a method for treating bipolar disorders which comprises treating a patient suffering therefore with a therapeutically effective amount of a compound (other than valproic acid) or a combination of compounds that is or are capable of stimulating the biochemical pathway for production and/or recycling of phosphatidylinositides such as PI, PIP and PIP₂. The compound should ideally be most active when activity in the PIns cycle is low.

From a fourth aspect the invention provides a means for screening a compound or combination of compounds for their suitability for use in treatment of bipolar disorder by testing for their ability to stimulate the biochemical pathway for production or recycling of phosphatidylinositides such as PI, PIP and PIP₂ and selecting a compound or combination of compounds effective to do this. The compound should ideally be most active when activity in the PIns cycle is low.

From a fifth aspect the present invention produces a method for treating bipolar disorders which comprises treating a patient suffering therefore with a therapeutically effective amount of a compound (other than valproic acid, carbamazepine and lithium) or a combination of compounds that is or are capable of inhibiting the biochemical pathway for production and/or recycling of phosphatidylinositides such as PI, PIP and PIP₂. The compound should ideally be most active when activity in the PIns cycle is high.

From a sixth aspect the invention provides a means for screening a compound or combination of compounds for their suitability for use in treatment of bipolar disorder by testing for their ability to inhibit the biochemical pathway for production and/or recycling of phosphatidylinositides such as PI, PIP and PIP₂ and selecting a compound or combination of compounds effective to do this. The compound should ideally be most active when activity in the PIns cycle is high.

Since extracellular myo-inositol can be transported into neurons as well as being recycled in it, from seventh and eight aspects, the present invention provides methods similar to the fifth and sixth, whereas rather than considering the ability of the compound selected to inhibit recycling of phosphatidylinositides using myo-inositol, one considers the ability of the compound to interfere with myo-inositol's ability to bond with a specific transporter in neurons or to prevent the translocation of such a transporter from the cytoplasm to the plasma membrane and insertion into the plasma membrane-see paper by Uldry et al., 2004. Such methods will exclude effects of compounds on the high-affinity sodium/myo-inositol transporters SMIT 1 and 2 that are also discussed in Uldry et al, 2004. The compound should ideally be most active when activity in the PIns cycle is high.

### BRIEF DESCRIPTION OF THE DRAWINGS

FIG. 1 is a diagram showing the key enzymes involved in the phosphoinositide (PIns) cycle.

FIG. 2 shows diagrams illustrating the compressor model of bipolar disorder and a summary of drug effects on the PIns cycle.

FIG. 3a and 3b present data showing the effect of desipramine and fluoxetine on the morphology of growth cones in the absence and in the presence of carbamazepine.

FIG. 4 presents data showing the effects of desipramine and fluoxetine on the morphology of growth cones in the absence and in the presence of valproic acid.

FIG. 5a presents data showing the effects of desipramine and fluoxetine on the morphology of growth cones in the presence of cpt-cAMP and compares these with the effect of cpt-cAMP and the PO inhibitor Z-pro-prolinal.

FIG. 5b presents data showing the effects of lithium, carbamazepine, valproic acid, cpt-cAMP combinations of the first four of these with cpt-cAMP and of the PO inhibitor Z-pro-prolinal on the response of growth cones.

FIG. 6a shows the effect of lithium on accumulation of CDP-DAG in cortical neurons stimulated with carbachol.

FIG. 6b shows the effect of inositol on lithium-induced accumulation of CDP-DAG in cortical neurons stimulated with carbachol. The inositol effect is not affected by the addition of 50 μM phloridzin.

FIG. 7 shows the effect of desipramine on lithium-induced accumulation of CDP-DAG in cortical neurons stimulated with carbachol.

FIG. 8 shows the effect of fluoxetine on accumulation of CDP-DAG in cortical neurons and the effect of inositol thereon.

FIG. 9 shows the effects of valproic acid, lithium, fluoxetine, desipramine, olanzepine and carbamazepine on prolyl oligopeptidase proteolytic activity. Only valproic acid inhibited the enzyme.

FIG. 10 shows the effect of the prolyl oligopeptidase inhibitor, Z-pro-prolinal, on lithium-induced accumulation of CDP-DAG in cortical neurons stimulated with carbachol.

FIG. 11a shows the effect of olanzapine on lithium-induced accumulation of CDP-DAG in cortical neurons stimulated with carbachol.

FIG. 11b shows the effect of tianeptine on lithium-induced accumulation of CDP-DAG in cortical neurons stimulated with carbachol.

FIG. 12 shows the effect of lamotrigine on the morphology of growth cones in the absence and in the presence of inositol or in the absence and in the presence of carbamazepine.

FIG. 13 shows the effect of tianeptine on the morphology of growth cones in the absence and in the presence of inositol or in the absence and in the presence of carbamazepine.

### DETAILED DESCRIPTION OF THE INVENTION

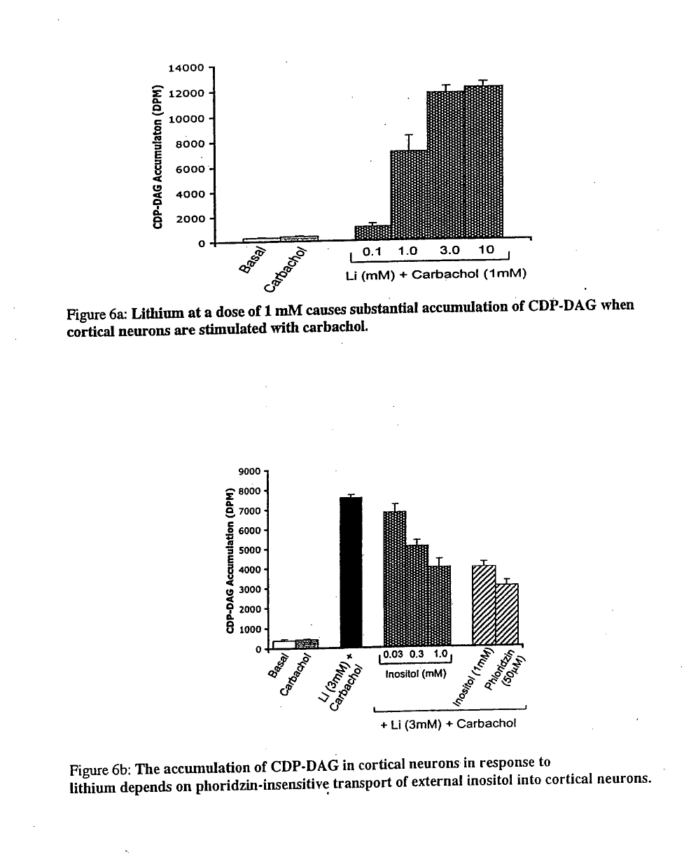

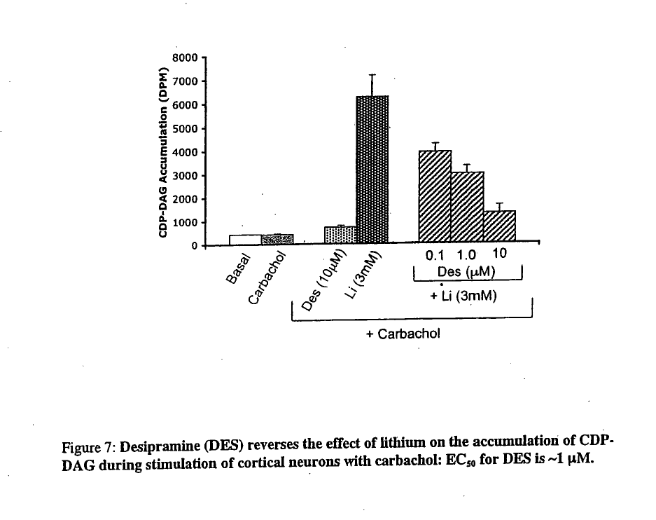

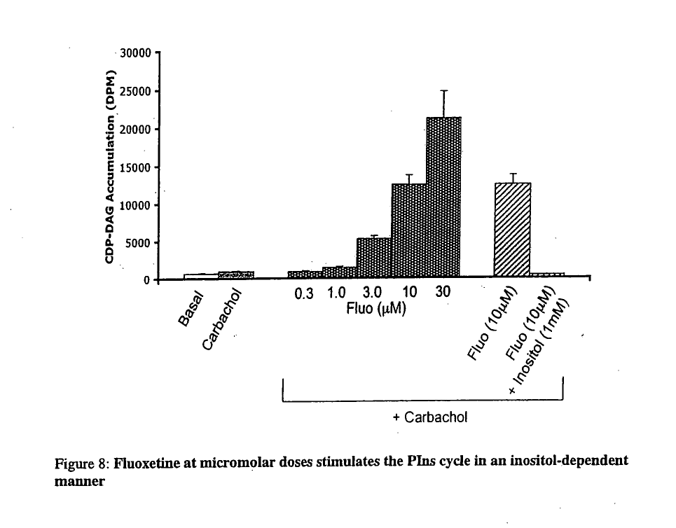

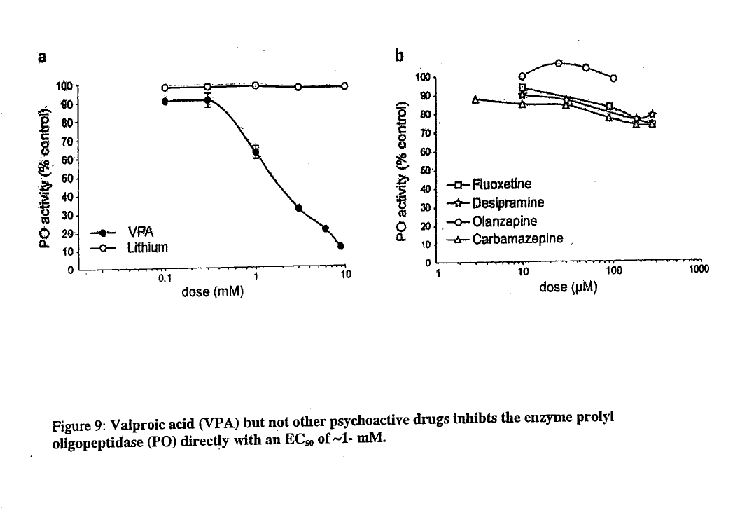

Inositol is not thought to be made by cortical neurons (Wong et al., 1987) but is very likely transported into them by high-affinity transporters such as HMIT recently described by (Uldry et al., 2004). Recent experiments in my laboratory confirm that at least some of the inositol used for synthesis or recycling of PIns in neurons is delivered by a transporter that is relatively resistant to drugs such as phloridzin know to inhibit SMITs at a concentration of 50 μM (Higgins and Kane, 2003). Phlorizdin at 50 μM did not affect the accumulation of ³H-CDP-DAG in cortical neurons treated with lithium (see FIG. 6b). Higher concentrations (5 mM) of phlorizin/phloridzin do inhibit HMIT, however (Uldry et al., 2001), suggeting that there may be some similarity, but not identity, in the phlorizdin-binding sites of SMIT and HMIT

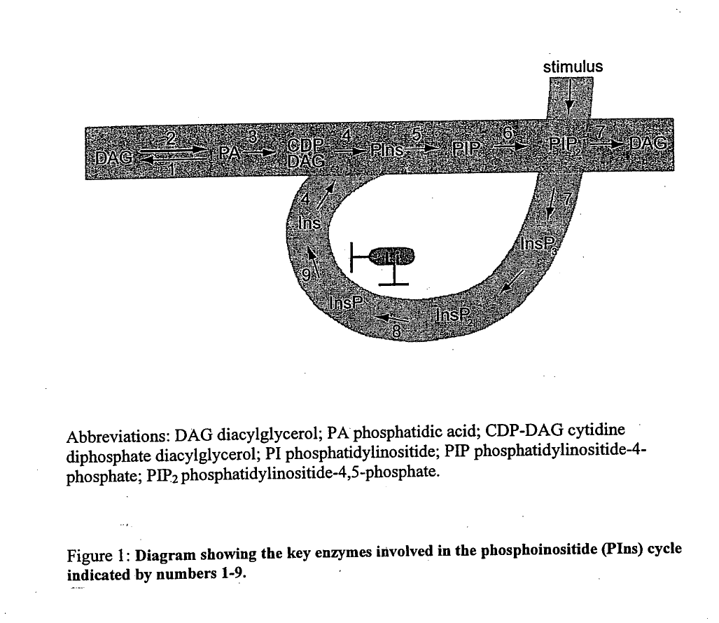

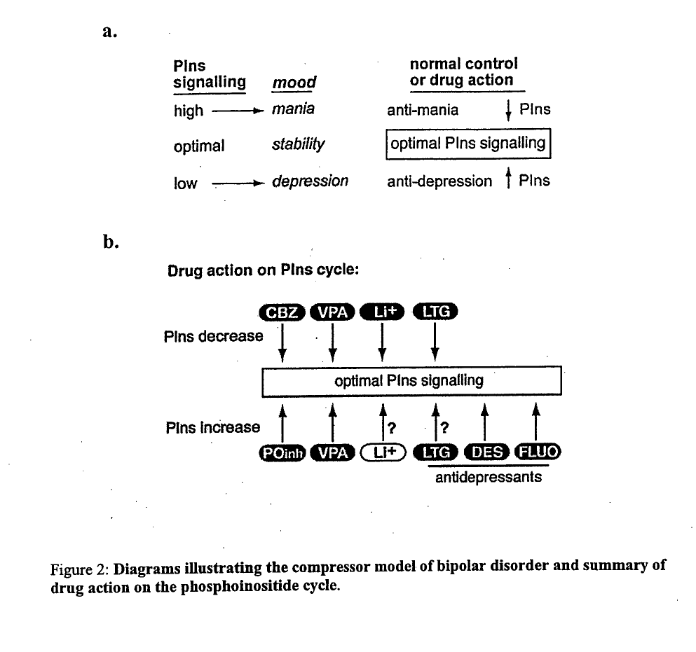

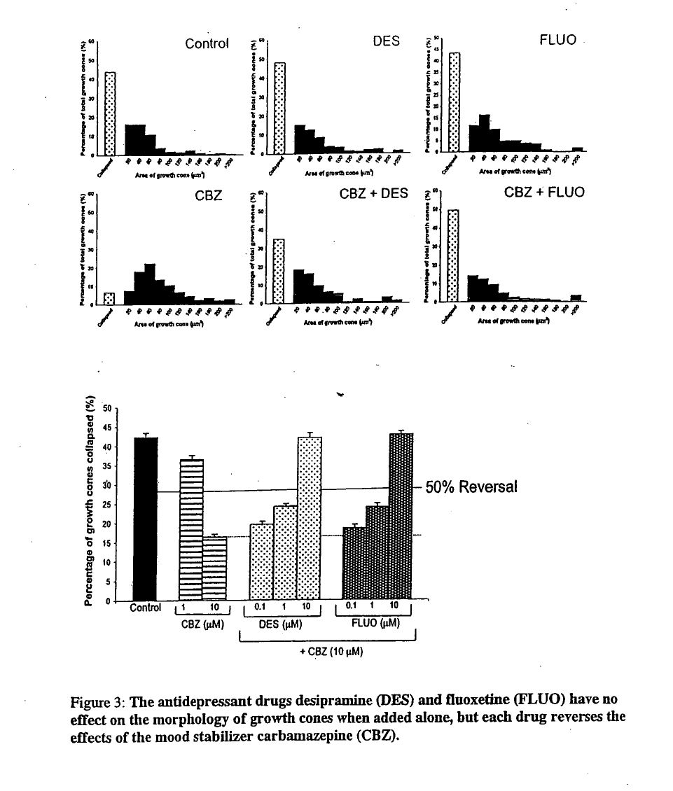

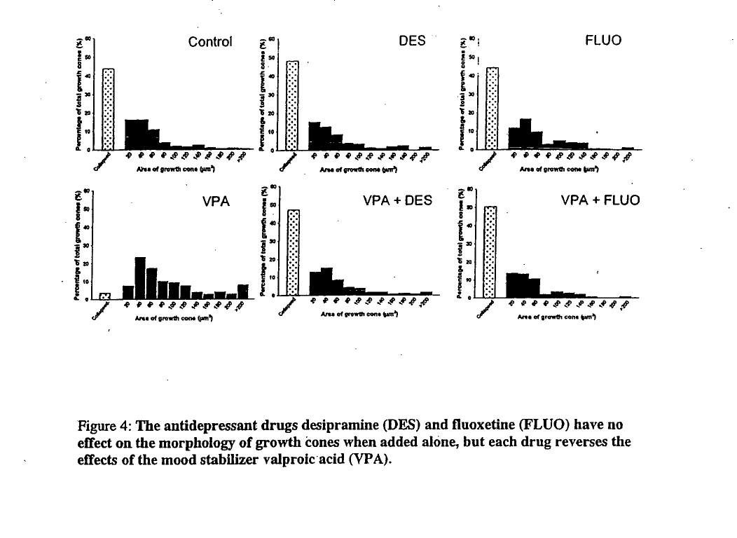

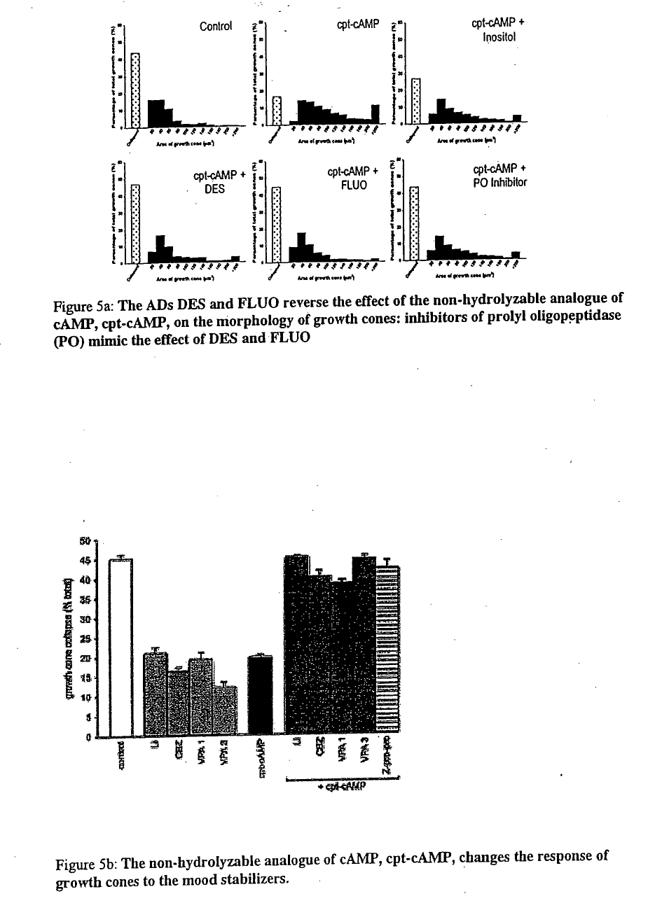

Inositol is itself transferred to cytidine diphosphate-diacylglycerol (CDP-DAG) to form phosphatidylinositol (PI), which is further phosphorylated by specific kinases (for which there may be specific neuronal forms) to form phosphatidylinositol monophosphate (PIP) and then further phosphorylated to form phosphatidylinositol-4,5-bisphosphate (PIP₂) in the cell membrane. This bisphosphate PIP₂ can itself bind many proteins necessary for synaptic function and thus regulate the function of these bound proteins and PIP₂ can be hydrolysed to form the second messengers in the PIns signalling pathway, namely, diacyl glyceral (DAG) and inositol-1,4,5-trisphosphate (InsP₃). InsP₃ is then dephosphorylated by a series of phosphatase enzymes such as inositol-polyphosphate-1-phosphatase (IPPase) and inositol monophosphatase (IMPase) to recycle myo-inositol. It has been thought that the recycled inositol is then used for synthesis of more PI, PIP and PIP₂ as discussed by Harwood (2005) and also by Berridge et al., (1989). Alternative possibilities are that the resynthesis of PIP₂ following an external stimulus that causes PIP₂ to be broken down uses myo-inositol, some of which may come from InsP₃ or other inositol polyphosphates (Shears, 2004) or some of which is supplied from extracellular sources. These alternate suggestions are consistent with new results obtained by me and my co-workers. (FIG. 1 details the enzyme steps).

Inhibition of the transport of myo-inositol into a neuron may be affected by finding a compound that will specifically interfere with the transport of myo-inositol into relevant neurons such as those in the frontal cortex of patients with bipolar disorder. Alternatively, since the translocation of some transporters are regulated in response to neuronal activity, this activity-dependent translocation and insertion into the membrane of suitable transporters or symporters would also be useful for the screening of drugs. Suitable cell lines such as described in Uldry et al., (2004) could be used to transfect and express the H+/myo-inositol transporter (HMIT) with a green-fluorescent protein (GFP) tag. Transport of tritiated inositol into the cells could be assessed in the presence or absence of compounds needing to be screened.

Suitable compounds for screening according to the present invention include antimanic drugs. Such compounds may be screened for by testing their ability to inhibit the transport of ³H-myo-inositol into cortical neurons in culture (which probably do not express the phloridzin-inhibitable SMITs)

Additionally, transport inhibitors may be tested using a neuronal cell line that expresses HMIT on the plasma membrane. Expression on the plasma membrane can also be achieved by transfecting cell lines with cDNA encoding HMIT with the ER retention signal deleted as described by Uldry et al. 2001, 2004. Assessing transport of myo-inositol is also well-described in these papers and is standard practice to those skilled in the art. Inhibitors found in either screen are then further tested using cortical neurons and determining whether the compounds of interest inhibit transport of myo-inositol into neurons and whether they inhibit PIns recycling. Screens that are of use include using tritiated inositol to measure its rate of uptake into cultured cortical neurons.

PIns recycling that is limited by the supply of inositol can be assessed by loading cells with ³H-cytidine, stimulating the cells with carbachol in the presence or absence of lithium, precipitating protein with trichloracetic acid, and then determining how much ³H accumulates as ³H-CDP-DAG in the lipid membrane compartment, which can be extracted with chloroform. A detailed method for this CDP-DAG assay is given in Gray et al (Gray et al., 1994) and such assays are the standard ones used to assess PIns recycling. Because HMIT is translocated to the plasma membrane in response to stimulation of PIns signalling, screening for compounds using HMIT should select for antimanic drugs that will be most active when there is increased PIns signalling. The same screen could be used to test compounds that inhibit translocation of HMIT to the membrane as this too will result in inhibition of transport. Since phloridzin and phloretin are weak inhibitors of HMIT but are more effective against SMIT, drugs based on the phoridzin or phloretin binding site but which specifically target the binding site of myo-inositol on HMIT and not other sugars on SMIT provide a good starting point. Compounds specifically for this purpose may be designed by a number of computer programs suitable for this purpose. When using such programs, the program will be run to design compounds having the following criteria: they will bind to HMIT at a dose less than 100 micromolar, but preferably less than 1 micromolar, they will be able to cross the blood-brain barrier, and they will inhibit the binding of myo-inositol in the range of 100-400 micromolar, which is that concentration of myo-inositol found in the CSF.

Another means of screening for compounds useful to treat the manic phase of bipolar disorder would be to test for their ability to inhibit the recycling of inositol from intracellular inositol polyphosphate stores by labelling them with H³-inositol and analysing the patterns of cytosolic inositol phosphates and membrane phoshoinositides: the novelty of this approach is to use cortical neurons rather than non-neural cells or neural cell lines.

Another means of screening for compounds useful to treat the manic phase of bipolar disorder would be to test for their ability to inhibit DAG-kinase and then to further screen for the ability of these compounds to inhibit the accumulation of CDP-DAG in response to carbachol stimulation of neurons in the presence of lithium as described above (see review of the DAG-kinase subtypes in (Luo et al., 2004). The assays for kinase activity where the substrates are normally in the lipid membrane bilayer require suspension of the enzyme and its substrate in a lipid micelle as is known to those expert in the field and described in Bunting et al., 1996, and Tang et al., 1996. Inhibitors for this should also work best when there is excess DAG to be recycled (eg when there is high signalling in the PIns pathway) and inhibitors showing uncompetitive inhibition kinetics should be searched for. In addition, phosphatidylinositol synthase and the kinases that convert PI to PIP and then to PIP₂, such as phosphatidylinositol kinase, phosphatidylinositol4-kinase, phosphatidylinositol-5-kinase, and phosphatidylinositol phosphate4-kinase and phosphatidylinositol phosphate-5-kinase are also suitable candidates for screening.

Testing the efficiency of compounds against the enzymes used in the pathway that generates PA, CDP-DAG, PI, PIP and PIP₂ can be effected by standard techniques known to those expert in the field. Typically purified recombinant enzymes can be obtained using a bacterial expression system with cDNA encoding the enzyme engineered so as to have a tag that can be used to purify the recombinant protein (see for example Cheng et al. 2005). cDNA encoding enzymes or transporters can often be obtained from the MRC's HGMP Resource Center and some can be obtained commercially. Alternatively, a membrane fraction can be obtained from say a neuronal-like cell line such as PC12 and enzyme activity assessed by adding labelled substrate and assessing the conversion of the substrate to a known product. Where possible, the type of enzymes that are known to be specifically localised to synapses should be used; for example, phosphatidylinositol phosphate kinase type 1γ (Di Paolo et al., 2002; Wenk et al., 2001). The enzyme and a substrate with which it can interact once placed in contact under suitable conditions and the degree of interaction is noted and the testing is then repeated in the presence of a candidate compound and the degree of inhibition under these circumstances also noted and compared with the results obtained in the absence of the candidate compound. The enzyme assays used to determine the activity of the enzymes are all in the prior art.

In order to find compounds useful in limiting the depressive pole of bipolar disorder, compounds that stimulate the PIns cycle are needed. Batty and Downes (Batty and Downes, 1995) showed that in some situations, CDP-DAG availability regulates the PIns cycle rather than inositol. When PIns signalling is low, then the availability of DAG may become limiting, thus leading to limiting amounts of CDP-DAG for recycling, so inhibiting phosphatidic acid phosphatase would increase PIns recycling. My research indicates that known antidepressant drugs may act in this way. A search should therefore aim to determine compounds that can inhibit phosphatidic acid phosphatase (PAPase) as this could increase the availability of PA and the PI precursor CDP-DAG. A first screen uses a conventional enzyme assay for phosphatase activity using the substrate PA and measuring release of phosphate with Malachite Green dye as is the convention. A secondary screen tests these compounds for their ability to stimulate PIns by assessing their ability to their ability to increase incorpopation of ³²P into PIP₂ in neurons. Suitable compounds for screening by include compounds related to fluoxetine, which likely inhibits PAPase, and compounds structurally related to other selective serotonin-reuptake inhibitors (SSRI).

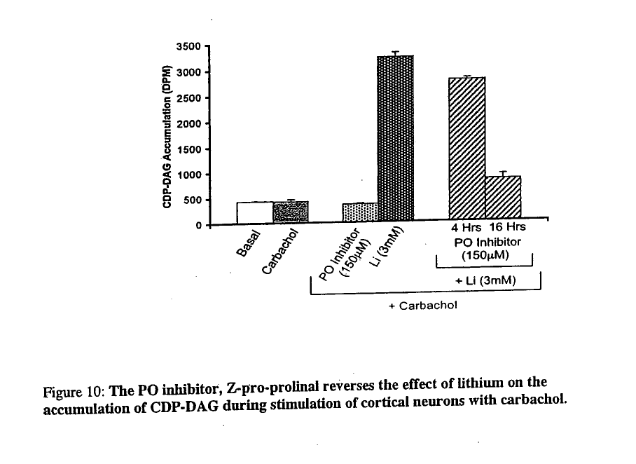

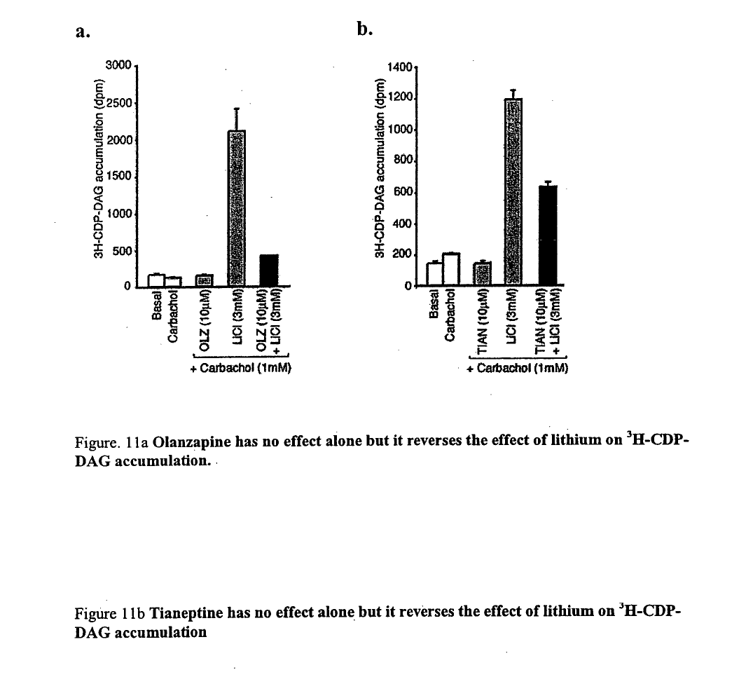

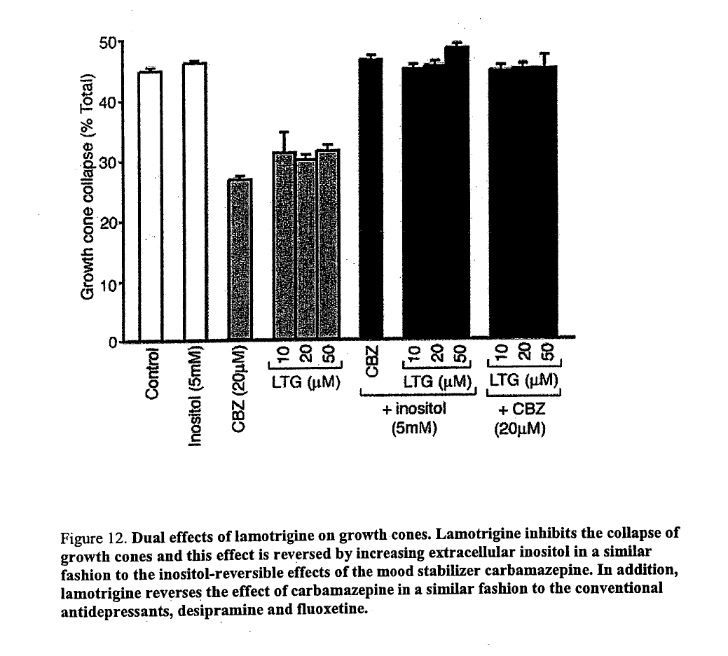

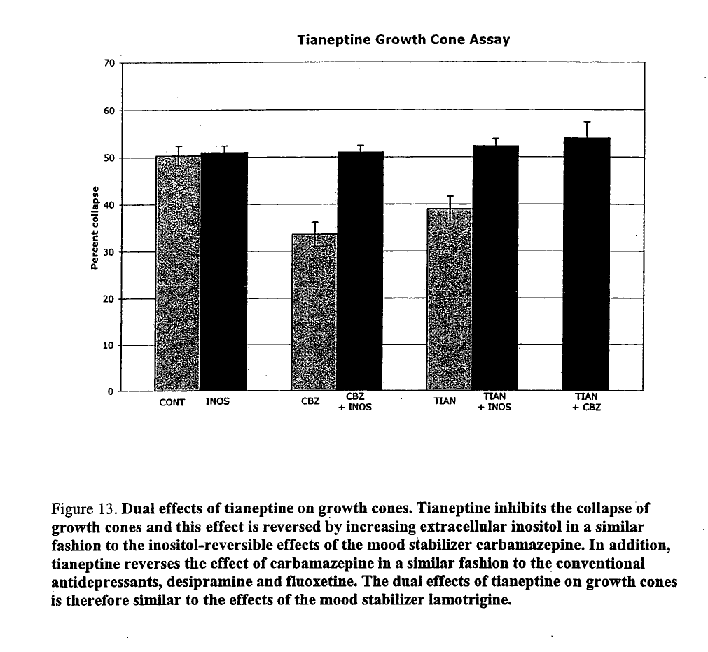

My research also indicates that known antidepressant drugs may act by increasing the availability of inositol required for phosphoinositide synthesis because a number of known antidepressant drugs can overcome the inhibitory effects of lithium on the synthesis of phosphoinositide. A suitable screen therefore will determine compounds that can reverse the effect of lithium as measured by the accumulation of CDP-DAG in carbachol-stimulated neurons. Another means of screening for drugs that reverse the effect of lithium on PIns recycling is to screen for drugs that alter the supply of intracellular inositol in neurons by labelling them with titriated inositol and analysing the patterns of cytosolic inositol phosphates and membrane phoshoinositides: the novelty of this approach is to use cortical neurons rather than non-neural cells or neural cell lines. Suitable compounds for screening include compounds related to tricyclic antidepressants such as desipramine and tianeptine, or to lamotrigine. Additional known tricyclics such as protriptyline that could also be tested. Selection of compounds that target stimulation of PIns synthesis but do not affect monoamine transport or other known monoamine receptors or compounds that are related to anticonvulsant drugs but do that do not mimic their anti-epileptic action would give the best specificity for bipolar disorder. I have shown that certain inhibitors of PO activity can mimic the activity of desipramine and reverse the inhibitory effect of lithium on the PIns cycle in neurons (see FIG. 10) and so these compounds pass the secondary screen outlined above for identifying compounds useful for treating the depressive phase of bipolar disorder. The same assays can be used to test for other compounds that stimulate PIns cycling including other inhibitors of prolyl oligopeptidase activity.

Another method of screening for suitable drugs for the prophylactic treatment of bipolar disorder is to look for compounds that have dual effects on the morphology of growth cones. Suitable compounds are those that can inhibit the collapse of growth cones in a way that is reversed by increasing extracellular inositol to 3 mM as well as reverse the effects of 20 μM carbamazepine or 3 mM valproic acid on the collapse of growth cones. Lamotrigine has such properties.

For inhibitors of the enzymatic activity on the enzyme prolyl oligopeptidase, the relevant references for several assays can be found in Cheng et al., MCN 2005. Common assays use a peptide-based substrate which, when cleaved from a prorlyl bond, releases a fluorogenic signal that can be quantitated by spectrocopy.

Suitable prolyl oligopeptidase inhibitors are those that readily penetrate into cells and which also exhibit a 50% inhibition of enzyme activity in the nanomolar to subnanomolar range. Those having an Kᵢ value of less than 100 nM more preferably less than 50 nM such as less than 20 nM, less than 10 nM or even less than 1 nM. Known inhibitors of prolyl oligopeptidase that meet these criteria include compounds S17092-1 (Portevin et al., 1996), JTP-4819 (Toide et al., 1995; Toide et al., 1997; Toide et al., 1998), SUAM 12221 (Saito et al., 1991) and ZTTA (see reference 9 in Portevin et al., 1996) Additional information on PO inhibitors is given in (Arai et al., 1993; Friedman et al., 1984; Morain et al., 2000; Tanaka et al., 1994; Toide et al., 1997; Toide et al., 1998; Wallen et al., 2002; Wilk and Orlowski, 1983).

Inhibitors of prolyl oligopeptidase may also bind to the beta-propeller of PO and so disrupt entrance of substrates to the proteolytic site. Inhibitors bound to the beta-propeller or other sites on may also interfere with protein-protein interactions without necessarily inhibiting its enzymatic activity. It is possible to use fluorescence spectroscopy to check for interference of candidate drugs with protein-protein interactions as described in (Berggard et al., 2002) and for drugs that change conformation of the PO beta-propellers as described in (Juhasz et al., 2005) or by disrupting its interaction with known proteins such as those described in Schulz et al., 2005.

Testing for suitable inhibitors of prolyl oligopeptidase activity that is also effective in stimulating synthesis of PIP₂ from within a cell may be carried out in a manner similar to that used for determining inhibition of PIns synthesis. The accumulation of tritiated CDP-DAG in the membrane fraction after loading cells with ³H-cytidine or the incorporation of ³²P orthophosphate into membrane phospholipids can be used to assess changes in synthetic patterns in the presence or absence of compounds needing to be tested. In some embodiments, the method may comprise determining whether the inhibition of enzyme activities or transporter activity or protein-protein interactions by the candidate compound is competitive and/or determining the amount of the compound needed to inhibit 50% of the activity of the enzyme (this IC50).

Conveniently, the effect of the candidate compounds that inhibit enzyme activity can be assessed using a labeled substrate that is cleaved or otherwise modified by prolyl oligopeptidase to produce a detachable change such as release of a label. Thus, it is possible to screen candidate inhibitors by measuring the change in the signal from the label caused by the action of prolyl oligopeptidase in the presence and absence of a candidate inhibitor compound. A preferred substrate may be one in which a fluorescent label is employed, such as Z-Gly-Pro-aminomethyl coumarin.

Conveniently, prolyl oligopeptidase enzyme assays can be carried out using a standard fluorogenic assay with the synthetic substrate Z-Gly-Pro-aminomethyl coumarin obtained from Bachem, Switzerland. Recominant prolyl oligopeptidase was produced in E. coli transfected with cDNA encoding the human form of prolyl oligopeptidase cloned into a bacterial expression vector containing either an N-or a C-terminal poly-histidine tag in order to purify the expressed protein. cDNA encoding human proline oligomerase can be obtained from the MRC's HGMP Resource Center, Hinxton Hall Cambridge, UK (Clone Image No. 3614248).

If desired a combination of the various screening methods described above may be used to determine the suitability of compounds for treatment of bipolar disorder.

Following identification of a candidate compound by one of the screens described above, the substance may be investigated further in order to determine its efficacy in penetrating the blood-brain-barrier and of entering into neurons by non-specific means, its cell permeability, toxicity, half-life and other standard testing to determine its suitability for formulation into a pharmaceutical composition.

The present invention also provides methods of treatment for bipolar disorders. Dosages for suitable agents can be determined by standard techniques such as injecting rats with various amounts of the compound of interest and then determining by assay how much has entered the brain and whether or not brain PO or other enzymes are inhibited. Extracts of brain can be subjected to chromatography procedures and amounts determined by standard methods, such as the use of mass spectroscopy.

I have now found that tianeptine, a tricyclic serotonin re-uptake stimulator (McEwen and Olie, 2005) effective in tests described above. Tianeptine is a modified tricyclic with strong antidepressant properties whose mechanisms of action has been a puzzle because it is a serotonin reuptake enhancer (McEwen and Olie, 2005) in contrast to fluoxetine, which is a serotonin reuptake inhibitor. When tested in the 3H-CDP-DAG assay tianeptine had no effect alone, but tianeptine abolished the lithium-induced accumulation of ³H-CDP-DAG in carbachol-stimulated neurons. Therefore it has stimulatory effects on the PIns cycle that are similar to desipramine. The effective doses were in the range of 10⁻⁶-10⁻⁵ M for tianeptine, consistent with their therapeutic plasma levels (Perry et al., 2001). The present invention therefore additionally provides a new means for treating bipolar disorder with tianeptine. Suitable dosages for use could be adapted from those currently in use to treat depression and initial trials could be done with patients suffering from bipolar depression

Formulations for use in the present invention typically are those used for oral administration and include tablets, capsules, caplets and other convenient devices. As noted above, the present invention may relate to the use of a single active component or a combination of actives that produce the specified results. If multiple active ingredients are employed, they may be formulated in a single dosage form or administered separately. Frequency of dosing will be determined by a physician using appropriate criteria. The best method of dosing will, however, be dependent on their half-life in the body and any effect that they may have on circadian rhythms.

### REFERENCES CITED

Agam, G., Shamir, A., Shaltiel, G., and Greenberg, M. L. (2002). Myo-inositol-1-phosphate (MIP) synthase: a possible new target for antibipolar drugs. Bipolar Disord 4 Suppl 1, 15-20.
Arai, H., Nishioka, H., Niwa, S., Yamanaka, T., Tanaka, Y., Yoshinaga, K., Kobayashi, N., Miura, N., and Ikeda, Y. (1993). Synthesis of prolyl endopeptidase inhibitors and evaluation of their structure-activity relationships: in vitro inhibition of prolyl endopeptidase from canine brain. Chem Pharm Bull (Tokyo) 41, 1583-1588.
Batty, I. H., and Downes, C. P. (1995). The mechanism of muscarinic receptor-stimulated phosphatidylinositol resynthesis in 1321N1 astrocytoma cells and its inhibition by Li⁺. J Neurochem 65, 2279-2289.
Berggard, T., Szczepankiewicz, O., Thulin, E., and Linse, S. (2002). myo-Inositol monophosphatase is an activated target of calbindin D28k. J Biol Chem 9, 9.
Berridge, M. J., Downes, C. P., and Hanley, M. R. (1989). Neural and developmental actions of lithium: a unifying hypothesis. Cell 59, 411-419.
Bunting, M., Tang, W., Zimmerman, G. A., McIntyre, T. M., and Prescott, S. M. (1996). Molecular cloning and characterization of a novel human diacylglycerol kinase zeta. J Biol Chem 271, 10230-10236.
Calabrese, J. R., Shelton, M. D., Rapport, D. J., and Kimmel, S. E. (2002a). Bipolar disorders and the effectiveness of novel anticonvulsants. J Clin Psychiatry 63, 5-9.
Calabrese, J. R., Shelton, M. D., Rapport, D. J., Kimmel, S. E., and Elhaj, O. (2002b). Long-term treatment of bipolar disorder with lamotrigine. J Clin Psychiatry 63 Suppl 10, 18-22.
Calabrese, J. R., Sullivan, J. R., Bowden, C. L., Suppes, T., Goldberg, J. F., Sachs, G. S., Shelton, M. D., Goodwin, F. K., Frye, M. A., and Kusumakar, V. (2002c). Rash in multicenter trials of lamotrigine in mood disorders: clinical relevance and management. J Clin Psychiatry 63, 1012-1019.
Di Paolo, G., Pellegrini, L., Letinic, K., Cestra, G., Zoncu, R., Voronov, S., Chang, S., Guo, J., Wenk, M. R., and De Camilli, P. (2002). Recruitment and regulation of phosphatidylinositol phosphate kinase type I gamma by the FERM domain of talin. Nature 420, 85-89.
Friedman, T. C., Orlowski, M., and Wilk, S. (1984). Prolyl endopeptidase: inhibition in vivo by N-benzyloxycarbonyl-prolyl-prolinal. J Neurochem 42, 237-241.
Fulop, V., Bocskei, Z., and Polgar, L. (1998). Prolyl oligopeptidase: an unusual beta-propeller domain regulates proteolysis. Cell 94, 161-170.
Goodwin, G. M. (2003). Evidence-based guidelines for treating bipolar disorder: recommendations from the British Association for Psychopharmacology. J Psychopharmacol 17, 149-173; discussion 147.
Gould, T. D., Quiroz, J. A., Singh, J., Zarate, C. A., and Manji, H. K. (2004). Emerging experimental therapeutics for bipolar disorder: insights from the molecular and cellular actions of current mood stabilizers. Mol Psychiatry 9, 734-755.
Gray, D. W., Challiss, R. A., and Nahorski, S. R. (1994). Differential effects of lithium on muscarinic cholinoceptor-stimulated CMP-phosphatidate accumulation in cerebellar granule cells, CHO-M3 cells, and SH-SY5Y neuroblastoma cells. J Neurochem 63, 1354-1360.
Guo, W., Shimada, S., Tajiri, H., Yamauchi, A., Yamashita, T., Okada, S., and Tohyama, M. (1997). Developmental regulation of Na⁺/myo-inositol cotransporter gene expression. Brain Res Mol Brain Res 51, 91-96.
Gurvich, N., and Klein, P. S. (2002). Lithium and valproic acid: parallels and contrasts in diverse signaling contexts. Pharmacol Ther 96, 45-66.
Harwood, A. J. (2005). Lithium and bipolar mood disorder: the inositol-depletion hypothesis revisited. Mol Psychiatry 10, 117-126.
Harwood, A. J., and Agam, G. (2003). Search for a common mechanism of mood stabilizers. Biochem Pharmacol 66, 179-189.
Higgins, B. D., and Kane, M. T. (2003). Inositol transport in mouse oocytes and preimplantation embryos: effects of mouse strain, embryo stage, sodium and the hexose transport inhibitor, phloridzin. Reproduction 125, 111-118.
Ju, S., Shaltiel, G., Shamir, A., Agam, G., and Greenberg, M. L. (2004). Human 1-D-myo-inositol-3-phosphate synthase is functional in yeast. J Biol Chem 279, 21759-21765.
Juhasz, T., Szeltner, Z., Fulop, V., and Polgar, L. (2005). Unclosed beta-propellers display stable structures: implications for substrate access to the active site of prolyl oligopeptidase. J Mol Biol 346, 907-917.
Luo, B., Regier, D. S., Prescott, S. M., and Topham, M. K. (2004). Diacylglycerol kinases. Cell Signal 16, 983-989.
Morain, P., Robin, J. L., De Nanteuil, G., Jochemsen, R., Heidet, V., and Guez, D. (2000). Pharmacodynamic and pharmacokinetic profile of S 17092, a new orally active prolyl endopeptidase inhibitor, in elderly healthy volunteers. A phase I study. Br J Clin Pharmacol 50, 350-359.
O'Brien, W. T., Harper, A. D., Jove, F., Woodgett, J. R., Maretto, S., Piccolo, S., and Klein, P. S. (2004). Glycogen synthase kinase-3beta haploinsufficiency mimics the behavioral and molecular effects of lithium. J Neurosci 24, 6791-6798.
Osborne, S. L., Meunier, F. A., and Schiavo, G. (2001). Phosphoinositides as key regulators of synaptic function. Neuron 32, 9-12.
Portevin, B., Benoist, A., Remond, G., Herve, Y., Vincent, M., Lepagnol, J., and De Nanteuil, G. (1996). New prolyl endopeptidase inhibitors: in vitro and in vivo activities of azabicyclo[2.2.2]octane, azabicyclo[2.2.1]heptane, and perhydroindole derivatives. J Med Chem 39, 2379-2391.
Post, R. M., Altshuler, L. L., Frye, M. A., Suppes, T., Rush, A. J., Keck, P. E., Jr., McElroy, S. L., Denicoff, K. D., Leverich, G. S., Kupka, R., and Nolen, W. A. (2001). Rate of switch in bipolar patients prospectively treated with second-generation antidepressants as augmentation to mood stabilizers. Bipolar Disord 3, 259-265.
Post, R. M., Baldassano, C. F., Perlis, R. H., and Ginsberg, D. L. (2003a). Treatment of bipolar depression. CNS Spectr 8, 1-10; quiz 11.
Post, R. M., Leverich, G. S., Nolen, W. A., Kupka, R. W., Altshuler, L. L., Frye, M. A., Suppes, T., McElroy, S., Keck, P., Grunze, H., and Walden, J. (2003b). A re-evaluation of the role of antidepressants in the treatment of bipolar depression: data from the Stanley Foundation Bipolar Network. Bipolar Disord 5, 396-406.
Saito, M., Hashimoto, M., Kawaguchi, N., Shibata, H., Fukami, H., Tanaka, T., and Higuchi, N. (1991). Synthesis and inhibitory activity of acyl-peptidyl-pyrrolidine derivatives toward post-proline cleaving enzyme; a study of subsite specificity. J Enzyme Inhib 5, 51-75.
Schulz, I., Gerhartz, B., Neubauer, A., Holloschi, A., Heiser, U., Hafner, M., and Demuth, H. U. (2002). Modulation of inositol 1,4,5-triphosphate concentration by prolyl endopeptidase inhibition. Eur J Biochem 269, 5813-5820.
Shaltiel, G., Shamir, A., Shapiro, J., Ding, D., Dalton, E., Bialer, M., Harwood, A. J., Belmaker, R. H., Greenberg, M. L., and Agam, G. (2004). Valproate decreases inositol biosynthesis. Biol Psychiatry 56, 868-874.
Shears, S. B. (2004). How versatile are inositol phosphate kinases? Biochem J 377, 265-280.
Tanaka, Y., Niwa, S., Nishioka, H., Yamanaka, T., Torizuka, M., Yoshinaga, K., Kobayashi, N., Ikeda, Y., and Arai, H. (1994). New potent prolyl endopeptidase inhibitors: synthesis and structure-activity relationships of indan and tetralin derivatives and their analogues. J Med Chem 37, 2071-2078.
Tang, W., Bunting, M., Zimmerman, G. A., McIntyre, T. M., and Prescott, S. M. (1996). Molecular cloning of a novel human diacylglycerol kinase highly selective for arachidonate-containing substrates. J Biol Chem 271, 10237-10241.
Toide, K., Iwamoto, Y., Fujiwara, T., and Abe, H. (1995). JTP-4819: a novel prolyl endopeptidase inhibitor with potential as a cognitive enhancer. J Pharmacol Exp Ther274, 1370-1378.
Toide, K., Shinoda, M., Iwamoto, Y., Fujiwara, T., Okamiya, K., and Uemura, A. (1997). A novel prolyl endopeptidase inhibitor, JTP-4819, with potential for treating Alzheimer's disease. Behav Brain Res 83, 147-151.
Toide, K., Shinoda, M., and Miyazaki, A. (1998). A novel prolyl endopeptidase inhibitor, JTPA4819—its behavioral and neurochemical properties for the treatment of Alzheimer's disease. Rev Neurosci 9, 17-29.
Uldry, M., Ibberson, M., Horisberger, J. D., Chatton, J. Y., Riederer, B. M., and Thorens, B. (2001). Identification of a mammalian H(+)-myo-inositol symporter expressed predominantly in the brain. Embo J 20, 4467-4477.
Uldry, M., Steiner, P., Zurich, M. G., Beguin, P., Hirling, H., Dolci, W., and Thorens, B. (2004). Regulated exocytosis of an H(+)/myo-inositol symporter at synapses and growth cones. Embo J 23, 531-540.
van Calker, D., and Belmaker, R. H. (2000). The high affinity inositol transport system—implications for the pathophysiology and treatment of bipolar disorder. Bipolar Disord 2, 102-107.
Wallen, E. A., Christiaans, J. A., Saario, S. M., Forsberg, M. M., Venalainen, J. I., Paso, H. M., Mannisto, P. T., and Gynther, J. (2002). 4-Phenylbutanoyl-2(S)-acylpyrrolidines and 4-phenylbutanoyl-L-prolyl-2(S)-acylpyrrolidines as prolyl oligopeptidase inhibitors. Bioorg Med Chem 10, 2199-2206.
Wenk, M. R., Pellegrini, L., Klenchin, V. A., Di Paolo, G., Chang, S., Daniell, L., Arioka, M., Martin, T. F., and De Camilli, P. (2001). PIP kinase Igamma is the major PI(4,5)P(2) synthesizing enzyme at the synapse. Neuron 32, 79-88.
Wilk, S., and Orlowski, M. (1983). Inhibition of rabbit brain prolyl endopeptidase by n-benzyloxycarbonyl-prolyl-prolinal, a transition state aldehyde inhibitor. J Neurochem 41, 69-75.
Williams, R. S., Cheng, L., Mudge, A. W., and Harwood, A. J. (2002). A common mechanism of action for three mood-stabilizing drugs. Nature 417, 292-295.
Williams, R. S., Eames, M., Ryves, W. J., Viggars, J., and Harwood, A. J. (1999). Loss of a prolyl oligopeptidase confers resistance to lithium by elevation of inositol (1,4,5) trisphosphate. Embo J 18, 2734-2745.
Wong, Y. H., Kalmbach, S. J., Hartman, B. K., and Sherman, W. R. (1987). Immunohistochemical staining and enzyme activity measurements show myo-inositol-1-phosphate synthase to be localized in the vasculature of brain. J Neurochem 48, 1434-1442.

1. A method for treating bipolar disorders which comprises treating a patient suffering therefore with a therapeutically effective amount of a cell-permeable compound (other than valproic acid) or a combination of compounds that is or are capable of inhibiting the activity of prolyl oligopeptidase within neurons.

2. A method for treating bipolar disorders which comprises treating a patient suffering therefore with a therapeutically effective amount of a compound (other than valproic acid or lamotrigine) or a combination of compounds that is or are capable of stimulating a biochemical pathway for phosphoinositide synthesis or recycling.

3. A method as claimed in claim 2, wherein said stimulation is effected by inhibiting phosphatidic acid phosphatase (using the subtypes specific for activity in the pathway used to synthesize PIns).

4. A method for treating bipolar disorders which comprises treating a patient suffering therefore with a therapeutically effective amount of a compound (other than valproic acid, lithium, carbamazepine, or lamotrigine) or a combination of compounds that is or are capable of inhibiting a biochemical pathway for phosphoinositide synthesis or recycling.

5. A method as claimed in claim 4, wherein said inhibition is effected by inhibiting at least one enzyme selected from diacylglycerol kinase, phosphatidylinositol synthase, phosphatidylinositol-4-kinase, phosphatidylinositol phosphate-5-kinase (using the subtypes specific for activity in the pathway used to synthesize PIns).

6. A method for treating bipolar disorders which comprises treating a patient suffering therefore with a therapeutically effective amount of a compound or a combination of compounds that is or are capable of inhibiting transport of myo-inositol into a neuron either by (1) inhibiting the transport of myo-inositol or by (2) preventing the translocation from the cytosol and/or insertion into the plasma membrane of a transporter or symporter that can transport myo-inositol from the extracellular space into a neuron.

7. A method for treating bipolar disorders which comprises treating a patient suffering therefore with a therapeutically effective amount of a compound compound (other than valproic acid, lithium, carbamazepine, or lamotrigine) or a combination of compounds that is or are capable of inhibiting the intracellular recycling of inositol polyphosphates to inositol.

8. A method of treating bipolar disorder which comprises administering a therapeutically effective amount of tianeptine to a patient suffering therefrom.

9. A method for screening a compound or combination of compounds for their suitability for use in treatment of bipolar disorder by testing for their ability to inhibit prolyl oligopeptidase within neurons and selecting a compound or combination of compounds effective for this.

10. A method for screening as claimed in claim 9, wherein said screen comprises use of a ³H-CDP-DAG on neurons that have been stimulated with lithium and carbachol or a ³H-inositol assay on neurons that have been stimulated with lithium and carbachol.

11. A method for screening a compound or combination of compounds for their suitability for use in treatment of bipolar disorder by testing for their ability to stimulate a biochemical pathway for phosphoinositide synthesis or recycling and selecting a compound or combination of compounds effective for this.

12. A method for screening as claimed in claim 11, wherein said screen comprises use of a ³H-CDP-DAG on neurons that have been stimulated with lithium and carbachol or a ³H-inositol assay on neurons that have been stimulated with lithium and carbachol.

13. A method for screening a compound or combination of compounds for their suitability for use in treatment of bipolar disorder by testing for their ability to inhibit a biochemical pathway for phosphoinositide synthesis or recycling and selecting a compound or combination of compounds effective for this.

14. A method of screening as claimed in claim 13, wherein said screen comprises use of a ³H-CDP-DAG on neurons that have been stimulated with and carbachol or a ³H-inositol assay on neurons that have been stimulated with carbachol.

15. A method as claimed in claim 13, wherein said inhibition is effected by inhibiting the transport of inositol by the H⁺/myo-inositol transporter or by inhibiting the translocation of this transporter to the plasma membrane.

16. A method as claimed in claim 15, wherein said screen comprises use of a ³H-inositol assay to assess transport into neurons.

17. A method for screening a compound or combination of compounds for their suitability for use in treatment of bipolar disorder by testing for their ability to inhibit transport of myo-inositol into a neuron either by (1) inhibiting the transport of myo-inositol or by (2) preventing the translocation from the cytosol and/or insertion into the plasma membrane of a transporter or symporter that can transport myo-inositol from the extracellular space into a neuron and selecting a compound or combination of compounds effective for this.

18. A method for screening a compound or combination of compounds for their suitability for use in treatment of bipolar disorder by testing for their ability to inhibit prolyl oligopeptidase within neurons and for their ability to stimulate a biochemical pathway for phosphoinositide synthesis or recycling and selecting a compound or combination of compounds effective to do both.

19. A method of screening compounds or a combination of compounds for their suitability for treatment of bipolar disorder which comprises a) measuring the release of phosphate when said compound or combination of compounds is employed in an assay of phosphatase activity on a phosphatidic acid and 2) screening said compound or combination of compounds by assaying their ability to reverse inhibition of PIns cycling in neurons by lithium or by their ability to increase incorporation of ³²P into PIP₂ in neurons.
